(19) United States
(12) Patent Application Publication
(10) Pub. No.: US 2018/0086852 A1
(43) Pub. Date: Mar. 29, 2018

(54) Hyaluronic Acid Derivative and Manufacturing Method Therefor and Cosmetics, Food Composition, and Pharmaceutical Composition Containing Hyaluronic Acid Derivative

(71) Applicant: Kewpie Corporation, Tokyo (JP)

(72) Inventors: Yukina Abe, Tokyo (JP); Hideto Yoshida, Tokyo (JP); Shunichi Fujikawa, Tokyo (JP); Atsushi Ohno, Tokyo (JP)

(73) Assignee: Kewpie Corporation, Tokyo (JP)

(21) Appl. No.: 15/559,268

(22) PCT Filed: Mar. 30, 2016
(86) PCT No.: PCT/JP2016/060525

§ 371 Date: Sep. 18, 2017

(30) Foreign Application Priority Data
Mar. 31, 2015 (JP) 2015-073753

(51) Int. Cl. C08B 37/08; A23L 33/10; A61K 8/73; A61Q 19/00; A61K 47/36

(52) U.S. Cl. CPC C08B 37/0072; A23L 33/10; A23V 2002/00; A61Q 19/00; A61K 47/36; A61K 8/735

(57) ABSTRACT

A method for manufacturing a hyaluronic acid derivative includes a step of reacting a carboxymethyl group-containing modified hyaluronic acid and/or a salt thereof with an organic compound containing an amino group and having a molecular weight of equal to or greater than 90.

### TECHNICAL FIELD

The present invention relates to a hyaluronic acid derivative, a manufacturing method thereof, and a cosmetic preparation, a food composition, and a pharmaceutical composition containing a hyaluronic acid derivative.

### BACKGROUND ART

In recent years, the modification of polysaccharides such as hyaluronic acid has been tried (Patent Literature 1). For example, for the purpose of altering the characteristics of polysaccharides or facilitating the intake of an organic compound into a biological body, the modification of polysaccharides with an organic compound has been tried.

### CITATION LIST

### Patent Literature

[Patent Literature 1] Japanese Unexamined Patent Publication No. H8-53501

### SUMMARY OF INVENTION

### Problems to be Solved by the Invention

The present invention provides a hyaluronic acid derivative of a modified hyaluronic acid and an organic compound, a manufacturing method of the hyaluronic acid derivative, and a cosmetic preparation, a food composition, and a pharmaceutical composition containing a hyaluronic acid derivative.

### Means for Solving the Problems

1. A method for manufacturing a hyaluronic acid derivative according to an aspect of the present invention includes a step of reacting a carboxymethyl group-containing modified hyaluronic acid and/or a salt thereof with an organic compound containing an amino group and having a molecular weight of equal to or greater than 90.

2. In the method for manufacturing a hyaluronic acid derivative described in 1, the organic compound may further contain a carboxyl group.

3. In the method for manufacturing a hyaluronic acid derivative described in 1 or 2, the amino group and the carboxyl group in the organic compound may be bonded to different carbon atoms.

4. In the method for manufacturing a hyaluronic acid derivative described in any one of 1 to 3, the amino group in the organic compound may be bonded to an alkylene group.

5. In the method for manufacturing a hyaluronic acid derivative described in any one of 1 to 4, the carboxymethyl group-containing modified hyaluronic acid and/or a salt thereof may have a constituent unit (1) shown below.

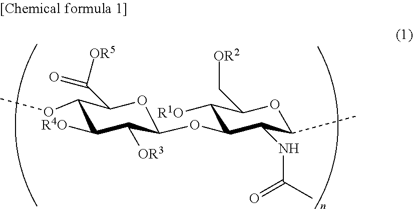

(In the formula, R¹, R², R³, R⁴, and R⁵ independently represent a hydrogen atom, a group represented by —CH₂—CO₂H, or a group represented by —CH₂—CO₂⁻, and n represents a number equal to or greater than 1 and equal to or less than 7,500 (here, a case is excluded where all of R¹, R², R³, R⁴, and R⁵ in the entirety of the carboxymethyl group-containing modified hyaluronic acid and/or a salt thereof represent a hydrogen atom).)

6. In the method for manufacturing a hyaluronic acid derivative described in any one of 1 to 5, a carboxymethylation rate with respect to a disaccharide unit constituting the carboxymethyl group-containing modified hyaluronic acid and/or a salt thereof may be equal to or higher than 5% and equal to or lower than 200%.

7. In the method for manufacturing a hyaluronic acid derivative described in any one of 1 to 6, the amino group contained in the organic compound may be a group represented by -NH₂.

8. A hyaluronic acid derivative according to an aspect of the present invention has a constituent unit (2) shown below.

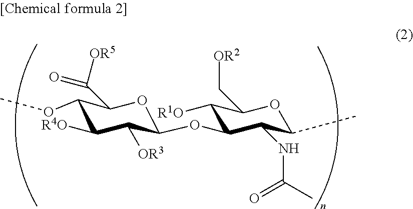

(In the formula, R¹, R², R³, R⁴, and R⁵ independently represent a hydrogen atom, a group represented by —CH₂—CO₂H, a group represented by —CH₂—CO₂⁻, or a group represented by Formula (3), and n represents a number equal to or greater than 1 and equal to or less than 7,500 (here, at least one of the groups represented by R¹, R², R³, R⁴, and R⁵ contained in the entirety of the hyaluronic acid derivative is a group represented by Formula (3)).)

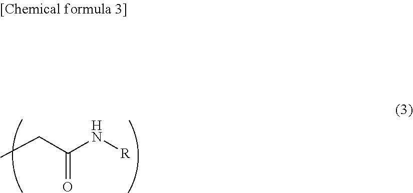

(In the formula, R represents an organic group having a molecular weight of equal to or greater than 74.)

9. In the hyaluronic acid derivative described in 8, a proportion of the group represented by Formula (3) contained in a disaccharide unit of a hyaluronic acid skeleton constituting the constituent unit (2) may be equal to or higher than 10%.

10. In the hyaluronic acid derivative described in 8 or 9, the constituent unit (2) may contain a group represented by —CH₂—CO₂H and/or a group represented by —CH₂^{—CO}₂⁻.

11. The hyaluronic acid derivative described in any one of 8 to 10 may be obtained by the method for manufacturing a hyaluronic acid derivative described in any one of 1 to 7.

12. A cosmetic preparation according to an aspect of the present invention contain the hyaluronic acid derivative described in any one of 8 to 11.

13. A food composition according to an aspect of the present invention contains the hyaluronic acid derivative described in any one of 8 to 11.

14. A pharmaceutical composition according to an aspect of the present invention contains the hyaluronic acid derivative described in any one of 8 to 11.

### Effects of the Invention

The method for manufacturing a hyaluronic acid derivative described in any one of 1 to 7 includes a step of reacting a carboxymethyl group-containing modified hyaluronic acid and/or a salt thereof with an organic compound containing an amino group and having a molecular weight of equal to or greater than 90. Accordingly, the organic compound can be efficiently bonded to the modified hyaluronic acid and/or a salt thereof.

The hyaluronic acid derivative described in any one of 8 to 11 has a moiety derived from the organic compound having a molecular weight of equal to or greater than 90. The hyaluronic acid derivative can be suitably used as a component of a cosmetic preparation, a food composition, and a pharmaceutical composition, for example.

### EMBODIMENTS FOR CARRYING OUT THE INVENTION

Hereinafter, the present invention will be specifically described. In the present invention, unless otherwise specified, “part” means “part by mass”, and “%” means “% by mass”.

[Hyaluronic Acid Derivative]

The hyaluronic acid derivative of the present invention refers to a compound obtained by bonding an organic compound containing an amino group and having a molecular weight of equal to or greater than 90 to a carboxymethyl group-containing modified hyaluronic acid and/or a salt thereof.

[Method for Manufacturing Hyaluronic Acid Derivative]

The method for manufacturing a hyaluronic acid derivative according to an embodiment of the present invention (hereinafter, simply described as “manufacturing method” in some cases) includes a step of reacting a carboxymethyl group-containing modified hyaluronic acid and/or a salt thereof (hereinafter, simply described as “modified hyaluronic acid” in some cases) with an organic compound containing an amino group and having a molecular weight of equal to or greater than 90 (hereinafter, simply described as “organic compound A” in some cases). Through the step of reaction, it is possible to obtain a hyaluronic acid derivative according to an embodiment which will be described later.

(Step of Reaction)

Through the aforementioned step of reaction, the organic compound A can be bonded to the modified hyaluronic acid. More specifically, through the step of reaction, the carboxyl group contained in the modified hyaluronic acid and the amino group in the organic compound A react with each other and form an amide bond, and as a result, the organic compound A can be bonded to the modified hyaluronic acid.

As a reagent used in the step of reaction, the reagents generally used at the time of peptide synthesis can be used. Examples of such reagents include a carbodiimide-based condensing agent such as 1-[3-(dimethylamino)propyl]-3-ethylcarbodiimide (EDC) and N,N′-dicyclohexylcarbodiimide (DCC), a triazine-based condensing agent such as 4-(4,6-dimethoxy-1,3,5-triazin-2-yl)-4-methylmorpholinium chloride n-hydrate (DMT-MM), an imidazole-based dehydrocondensing agent such as N,N′-carbonyldiimidazole (CDI), a phosphonium-based condensing agent such as 1H-benzotriazol-1-yloxytris(dimethylamino)phosphonium hexafluorophosphate (BOP), a uronium-based condensing agent such as {{[(1-cyano-2-ethoxy-2-oxoethylidene)amino]oxy}-4-morpholinometh ylene}dimethyl ammonium hexafluorophosphate (COMU), and the like. Furthermore, additives for a condensation reaction such as 1-hydroxybenzotriazole (HOBt), 1-hydroxyazabenzotriazole (HOAt), or N-hydroxysuccinimide (NHS) can be used in combination with the aforementioned condensing agents.

The ratio between the reagent and the modified hyaluronic acid (molar ratio: reagent/modified hyaluronic acid) is generally equal to or higher than 1/100 and equal to or lower than 3/1, and the ratio between the modified hyaluronic acid and the organic compound A (molar ratio: modified hyaluronic acid/organic compound A) is generally equal to or higher than 1/100 and equal to or lower than 4/1.

The reaction time in the step of reaction is generally equal to or longer than 1 hour and equal to or shorter than 100 hours (preferably equal to or longer than 1 hour and equal to or shorter than 50 hours). The reaction temperature in the step of reaction is generally equal to or higher than 4° C. and equal to or lower than 100 ° C. (preferably equal to or higher than 10° C. and equal to or lower than 80° C.). The pH of the reaction solution in the step of reaction is generally equal to or higher than 1 and equal to or lower than 12 (preferably equal to or higher than 3 and equal to or lower than 10).

(Raw Material: Modified Hyaluronic Acid)

The molecular weight of the modified hyaluronic acid and/or a salt thereof used as a raw material (hereinafter, for being distinguished from “raw material hyaluronic acid”, described as “raw material modified hyaluronic acid” in some cases) in the manufacturing method according to the present embodiment is generally equal to or greater than 800 and equal to or less than 4,000,000. For example, the molecular weight is preferably equal to or greater than 2,000, and more preferably equal to or greater than 3,000. In contrast, the molecular weight is preferably equal to or less than 1,000,000, more preferably equal to or less than 600,000, and even more preferably equal to or less than 50,000. The molecular weight of the modified hyaluronic acid can be measured by the following method.

By using a gel filtration column, a plurality of hyaluronic acids (standard substances) whose molecular weight is known are analyzed by liquid chromatography, and from the retention time thereof, a calibration curve is plotted. Likewise, by analyzing the modified hyaluronic acid to be measured by liquid chromatography and determining the molecular weight by using the calibration curve, the molecular weight of the modified hyaluronic acid can be determined.

Examples of the liquid chromatography analyzer which can be used for the aforementioned liquid chromatography analysis include Waters Alliance 2690 HPLC Separations Module (manufactured by WATERS), Waters Alliance 2695 HPLC separations Module (manufactured by WATERS), and a 1200 Series (manufactured by Agilent Technologies). Examples of the column which can be used for the liquid chromatography analysis include columns for ligand exchange chromatography (ligand exchange mode+size exclusion mode) manufactured by Shodex with model names of “SUGAR KS-801”, “SUGAR KS-802”, “SUGAR KS-803”, “SUGAR KS-804”, “SUGAR KS-805”, “SUGAR KS-806”, and “SUGAR KS-807”, and a size exclusion chromatography column manufactured by Tosoh Corporation with a model name of “TSKgel GMPW”.

The carboxymethylation rate of the modified hyaluronic acid can be equal to or higher than 5% and is preferably equal to or higher than 10% and more preferably equal to or higher than 20%. The carboxymethylation rate may be equal to or higher than 30%, for example. Furthermore, the carboxymethylation rate can be equal to or lower than 200%, and is more preferably equal to or lower than 150%. For example, the carboxymethylation rate may be equal to or lower than 100% and equal to or lower than 85%.

In the manufacturing method according to the present embodiment, there is a correlation between the carboxymethylation rate of the modified hyaluronic acid and the modification rate of the organic compound A. The higher the carboxymethylation rate of the modified hyaluronic acid is, the higher the modification rate tends to be. Presumably, for this reason, the organic compound A may mainly react with the carboxymethyl group.

In the present invention, the carboxymethylation rate of the modified hyaluronic acid is represented by a ratio (%) of an integrated value of peaks (appear within a range of 3.8 ppm to 4.2 ppm) showing a proton of a methylene group (—CH₂—) in the group represented by —CH₂—CO₂H and/or —CH₂—CO₂₋ to an integrated value of peaks (appear at around 2 ppm) showing a proton of a methyl group (—CH₃) of a N-acetyl group bonded to the C-2 position in the hyaluronic acid skeleton in a ¹H-NMR spectrum.

(Method for Manufacturing Modified Hyaluronic Acid)

The modified hyaluronic acid used in the manufacturing method according to the present embodiment can be manufactured by the following method, for example.

(Raw Material Hyaluronic Acid and/or Salt Thereof)

In the present invention, “hyaluronic acid” refers to polysaccharides having one or more repeating constituent units formed of disaccharide consisting of glucuronic acid and N-acetylglucosamine Although “salt of hyaluronic acid” is not particularly limited, it is preferably a sitologically and pharmaceutically acceptable salt. Examples thereof include a sodium salt, a potassium salt, a calcium salt, a zinc salt, a magnesium salt, an ammonium salt, and the like.

Basically, the hyaluronic acid is a substance composed of two or more sugar molecules containing at least one disaccharide unit in which the 1-position of β-D-glucuronic acid is bonded to the 3-position of β-D-N-acetyl glucosamine The hyaluronic acid is basically constituted with β-D-glucuronic acid and β-D-N-acetyl glucosamine, in which a plurality of disaccharide units are bonded to each other. The sugar may be unsaturated sugar, and examples of the unsaturated sugar include sugars with nonreducing terminals, generally, sugars in which the bond between carbon atoms in 4- and 5-positions of glucuronic acid is unsaturated, and the like.

The raw material hyaluronic acid and/or a salt thereof (hereinafter, simply described as “raw material hyaluronic acid” in some cases) used for manufacturing the modified hyaluronic acid according to the present embodiment may be extracted from natural substances from animals (for example, biological tissue such as cock's comb, the umbilical cord, the skin, and the synovial fluid). Furthermore, as the raw material hyaluronic acid, it is possible to use those obtained by the culture of microorganisms, animal cells, or plant cells (for example, a fermentation method using bacteria of genus Staphylococcus) or those chemically or enzymatically synthesized.

As the raw material hyaluronic acid, both the crude extract and the purified substance may be used. However, it is preferable to use the purified substance, specifically, the raw material hyaluronic acid with a purity of 90% (mass ratio), because then the carboxymethylation can smoothly proceed.

(Average Molecular Weight of Raw Material Hyaluronic Acid)

In the manufacturing method according to the present embodiment, the average molecular weight of the raw material hyaluronic acid dissolved during the step of reaction is preferably equal to or greater than 4,000 and equal to or less than 4000,000 in general, and more preferably equal to or less than 3,000,000, because then the carboxymethylation can smoothly proceed. The average molecular weight of the raw material hyaluronic acid can be measured by the following method.

(Method for Measuring Molecular Weight)

That is, about 0.05 g of (purified) hyaluronic acid (original substance) is accurately weighed and dissolved in a sodium chloride solution with a concentration of 0.2 mol/L so as to make a solution with a volume of exactly 100 mL. The solution is accurately weighed out at 8 mL, 12 mL, and 16 mL, and a sodium chloride solution with a concentration of 0.2 mol/L is added to each of the solutions so as to make solutions with a volume of 20 mL. The solutions are used as sample solutions. For the sample solutions and the sodium chloride solution with a concentration of 0.2 mol/L, specific viscosity is measured (Equation A) at 30.0±0.1° C. by the viscosity measurement method (Method I viscosity measurement by capillary tube viscometer) in General Tests in the Japanese Pharmacopoeia (16^{th} edition), and reduced viscosity at each concentration is calculated (Equation (B)). Then, a graph is drawn by plotting the reduced viscosity on the ordinate and the concentration (g/100 mL) of the original substance expressed in terms of the dried substance on the abscissa. From the intersection point between a straight line connecting the respective points and the ordinate, limiting viscosity is determined. By plugging the limiting viscosity determined in this way into the Laurent's equation (Equation (C)), the average molecular weight is calculated (Torvard C Laurent, Marion Ryan, and Adolph Pietruszkiewicz, “Fractionation of hyaluronic Acid”, Biochemical et Biophysical Acta., 42, 476-485 (1960), Chikako Yotoma, “Molecular weight evaluation of sodium hyaluronate preparation by SEC-MALLS”, the Journal of National Institute of Health Sciences, No. 121, 030-033 (2003)).

Specific viscosity={time taken for sample solution to flow down (sec)}/(time taken for 0.2 mol/L sodium chloride solution to flow down (sec)}−1 (Equation A)

Reduced viscosity (dL/g)=specific viscosity/(concentration of original substance expressed in terms of dried substance (g/100 mL)) (Equation B)

Limiting viscosity (dL/g)=3.6×10⁻⁴M^{0.78}M: average molecular weight (Equation C)

(Content of Raw Material Hyaluronic Acid)

The content of raw material hyaluronic acid in the raw material hyaluronic acid is a parameter of the purity of the raw material hyaluronic acid. It can be mentioned that the greater the content of raw material hyaluronic acid, the higher the purity of the raw material hyaluronic acid.

In the present invention, the content of hyaluronic acid in the raw material hyaluronic acid is a value calculated from the quantity of glucuronic acid measured by a carbazole-sulfuric acid method (for example, the Japanese Pharmacopoeia).

The carbazole-sulfuric acid method is a method in which an aqueous hyaluronic acid solution is added to and mixed with a sodium borate·sulfuric acid solution, the hyaluronic acid is decomposed by heating and then cooled, a carbazole·ethanol solution is added to and mixed with the solution, the resulting solution is heated and then left to cool, and an absorbance (530 nm) of the obtained sample solution is measured. By using D-glucuronolactone treated in the same manner, a calibration curve is plotted, and a value expressed in terms of D-glucuronolactone is calculated. Then, the calculated value is multiplied by 1.102, thereby determining the quantity of glucuronic acid. The determined quantity of glucuronic acid is multiplied by (molecular weight of hyaluronic acid/molecular weight of glucuronic acid), thereby calculating the content of the hyaluronic acid.

(Carboxymethylation)

In the present invention, “carboxymethyl group” refers to a group represented by “—CH₂—CO₂H” or “—CH₂—CO₂⁻”. Therefore, in the present invention, “carboxymethyl group-containing modified hyaluronic acid and/or a salt thereof”refers to a hyaluronic acid into which a carboxymethyl group is introduced in at least a portion and/or a salt thereof.

More specifically, in the modified hyaluronic acid according to the present embodiment, for example, hydrogen atoms of at least some of hydroxyl groups (in Formula (4), the C-4 and C-6 positions in the N-acetylglucosamine constituting the hyaluronic acid and the C-2 and C-3 positions in the glucuronic acid constituting the hyaluronic acid) constituting the hyaluronic acid (see Formula (4)) as a raw material can be substituted with a group represented by —CH₂—CO₂H and/or —CH₂—CO₂⁻. That is, in the modified hyaluronic acid according to the present embodiment, the hydrogen atoms of the hydroxyl groups in one or two or more positions among the hydroxyl groups in the above positions may be substituted with the group represented by —CH₂—CO₂H and/or —CH₂—CO₂⁻.

In the present invention, “disaccharide unit of the hyaluronic acid skeleton” refers to a single constituent unit which constitutes a hyaluronic acid and is constituted with two neighboring sugar molecules (glucuronic acid and N-acetylglucosamine) bonded to each other, and “carboxymethylation rate with respect to the disaccharide unit of the hyaluronic acid skeleton” is the number of carboxymethyl groups contained in the single constituent unit. More specifically, in a case where the single constituent unit is regarded as being 100%, the carboxymethylation rate refers to a proportion (%) of the number of carboxymethyl groups contained in the single constituent unit.

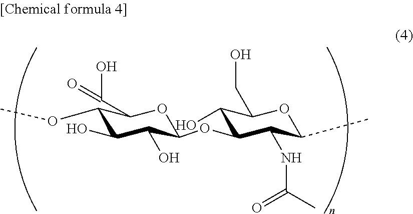

(In the formula, n represents a number equal to or greater than 1 and equal to or less than 7,500.)

The raw material modified hyaluronic acid used in the manufacturing method according to the present embodiment can have a constituent unit represented by Formula (1), for example. In the hyaluronic acid derivative according to the present embodiment, at least some of R²'s in the raw material modified hyaluronic acid are preferably a group represented by —CH₂—CO₂H or a group represented by —CH₂—CO₂⁻, because then the reactivity with the organic compound A is further improved.

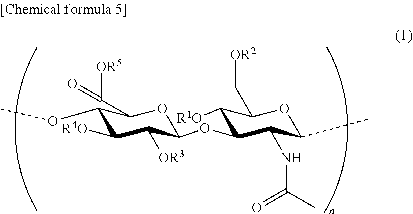

(In the formula, R¹, R², R³, R⁴, and R⁵ independently represent a hydrogen atom, a group represented by —CH₂—CO₂H, or a group represented by —CH₂—CO₂⁻, and n represents a number equal to or greater than 1 and equal to or less than 7,500 (herein, a case is excluded where all of R¹, R², R³, R⁴, and R⁵ in the entirety of the raw material modified hyaluronic acid represent a hydrogen atom).)

(pH)

In the manufacturing method according to the present embodiment, the reaction is preferably performed under the basic condition, and the pH of the reaction solution (water-containing solvent) is more preferably equal to or higher than 9 (equal to or higher than 9 and equal to or lower than 14, preferably equal to or higher than 10 and equal to or lower than 14, and even more preferably equal to or higher than 11 and equal to or lower than 14), because then the nucleophilicity of a hydroxyl group can be improved.

In order to make the reaction solution basic, a basic electrolyte can be used in the reaction solution. Examples of the basic electrolyte include a hydroxide of an alkali metal such as sodium hydroxide and potassium hydroxide, and a hydroxide of an alkali earth metal such as calcium hydroxide, magnesium hydroxide, and barium hydroxide. The concentration of the basic electrolyte in the reaction solution is equal to or higher than 0.2 mol/L and equal to or lower than 10 mol/L for example, and preferably equal to or higher than 0.5 mol/L and equal to or lower than 8 mol/L, because then the modified hyaluronic acid of all of a first manufacturing example and a second manufacturing example, which will be described later, can be efficiently obtained.

Furthermore, the concentration of the hyaluronic acid in the water-containing solvent is preferably equal to or higher than 0.05 g/mL and equal to or lower than 0.5 g/mL, because then the modified hyaluronic acid of all of the first manufacturing example and the second manufacturing example, which will be described later, can be efficiently obtained.

(Haloacetic Acid and/or Salt Thereof)

In the method for manufacturing a modified hyaluronic acid according to the present embodiment (hereinafter, simply described as “manufacturing method according to the present embodiment” in some cases), either or both of haloacetic acid and a salt thereof are used for introducing a carboxymethyl group into the raw material hyaluronic acid and/or a salt thereof.

The haloacetic acid can be a monohaloacetic acid and/or a salt thereof, for example. More specifically, the haloacetic acid is preferably chloroacetic acid and/or a salt thereof or bromoacetic acid and/or a salt thereof. For example, a salt of the haloacetic acid is preferably an alkali metal salt of chloroacetic acid and/or an alkali metal salt of bromoacetic acid, and more preferably sodium chloroacetate and/or sodium bromoacetate.

(Amount of Haloacetic Acid and/or Salt Thereof Used)

The amount of the haloacetic acid and/or salt thereof used is generally equal to or greater than 10% and equal to or less than 500% (mass ratio) and preferably equal to or greater than 50% and equal to or less than 200% (mass ratio) of the amount of the raw material hyaluronic acid and/or a salt thereof used.

(Water-Containing Solvent)

In the manufacturing method according to the present embodiment, in a case where the water-containing solvent is water or a mixed solution of a water-soluble organic solvent and water, the solvent excellently dissolves the raw material hyaluronic acid and/or a salt thereof.

In a case where the water-containing solvent is a mixed solution of a water-soluble organic solvent and water, that is, in a case where the water-containing solvent containing both water and the water-soluble organic solvent, the proportion of the water-soluble organic solvent in the water-containing solvent is generally equal to or lower than 60 v/v % (higher than 0 v/v % and equal to or lower than 60 v/v %) and preferably equal to or lower than 40 v/v % (higher than 0 v/v % and equal to or lower than 40 v/v %), because then the solubility of the hyaluronic acid can be improved.

Examples of the water-soluble organic solvent include an alcohol-based solvent such as methanol, ethanol, 1-propanol, and 2-propanol, a ketone-based solvent such as acetone and methyl ethyl ketone, tetrahydrofuran, acetonitrile, and the like. These solvents can be used singly or used in combination. Among these, lower alcohols having 1, 2, or 3 carbon atoms such as isopropanol and ethanol are preferable.

(Reaction Temperature)

In the aforementioned reaction, the temperature of the reaction solution is preferably equal to or lower than 30° C. (preferably higher than 0° C. and equal to or lower than 30° C.) in general, and more preferably equal to or lower than 10° C. (preferably higher than 0° C. and equal to or lower than 10° C.), because then the carboxylation can smoothly proceed and the reduction in the molecular weight can be inhibited. Particularly, in a case where the temperature of the reaction solution is equal to or lower than 10° C., a modified hyaluronic acid having a high molecular weight (equal to or greater than 800,000) can be obtained in a simple manner.

For example, in a case where either or both of chloroacetic acid and a salt thereof are used as the haloacetic acid and/or a salt thereof, the temperature of the reaction solution in the reaction can be set to be a general temperature (preferably higher than 0° C. and equal to or lower than 30° C.) and is preferably equal to or higher than 1° C. and equal to or lower than 30° C., because then the carboxymethylation can smoothly proceed and the browning of the obtained modified hyaluronic acid can be inhibited.

Furthermore, for example, in a case where either or both of bromoacetic acid and a salt thereof are used as the haloacetic acid and/or a salt thereof, the temperature of the reaction solution in the reaction can be equal to or lower than 10° C. (preferably higher than 0° C. and equal to or lower than 10° C.) in general and is preferably equal to or higher than 1° C. and equal to or lower than 10° C., because then the carboxymethylation can smoothly proceed and the browning and the molecular weight reduction of the obtained modified hyaluronic acid can be inhibited.

More specifically, in order to manufacture a modified hyaluronic acid having a high molecular weight (for example, a molecular weight of equal to or greater than 800,000) and a high (for example, equal to or higher than 5% and preferably equal to or higher than 5% and equal to or lower than 200%) carboxymethylation rate with respect to the disaccharide unit of the hyaluronic acid skeleton (hereinafter, simply referred to as “carboxymethylation rate” as well) as in the first manufacturing example which will be described later, it is preferable to perform the reaction by using bromoacetic acid and/or a salt thereof as the haloacetic acid and/or a salt thereof at a temperature of the reaction solution of equal to or lower than 10° C. (for example, higher than 0° C. and equal to or lower than 10° C.).

In order to manufacture a modified hyaluronic acid having a low molecular weight (for example, a molecular weight of less than 800,000) and a high (for example, equal to or higher than 30% and preferably equal to or higher than 30% and equal to or lower than 200%) carboxymethylation rate as in the second manufacturing example which will be described later, it is preferable to perform the reaction at a temperature of the reaction solution of equal to or higher than 10° C. (for example, equal to or higher than 10° C. and equal to or lower than 35° C., preferably equal to or higher than 15° C., more preferably equal to or higher than 20° C., and even more preferably room temperature).

(Reaction Time)

In the aforementioned reaction, the reaction time is preferably equal to or longer than 30 minutes and equal to or shorter than 100 hours in general and more preferably equal to or longer than 60 minutes and equal to or shorter than 60 hours, because then the carboxylation can smoothly proceed and the reduction in the molecular weight can be inhibited.

First Manufacturing Example

By the aforementioned manufacturing method, a modified hyaluronic acid having a molecular weight of equal to or greater than 800,000 can be obtained in a simple manner. That is, according to the manufacturing method of the present embodiment, it is possible to obtain a modified hyaluronic acid having a high molecular weight and high whiteness in a simple manner.

In this case, the carboxymethylation rate of the obtained modified hyaluronic acid can be equal to or higher than 5% and equal to or lower than 200%.

Second Manufacturing Example

Alternatively, by the aforementioned manufacturing method, it is possible to obtain a modified hyaluronic acid having a molecular weight of equal to or greater than 800 and less than 800,000 in a simple manner.

In this case, the carboxymethylation rate of the obtained modified hyaluronic acid can be equal to or higher than 30% and equal to or lower than 200%. That is, by the aforementioned manufacturing method, it is possible to obtain a modified hyaluronic acid having a relatively low molecular weight, which is equal to or greater than 800 and less than 800,000, a high carboxymethylation rate which is equal to or higher than 30% and equal to or lower than 200%, and high whiteness in a simple manner.

(Raw Material: Organic Compound A)

In the hyaluronic acid derivative according to an embodiment which will be described later, the organic compound A becomes a portion which is bonded as an organic group (for example, a group represented by R in a group represented by Formula (2) which will be described later) to the disaccharide unit of the hyaluronic acid skeleton. In a case where the organic compound A has an amino group, in the aforementioned step of reaction, the compound can be bonded to the disaccharide unit of the hyaluronic acid skeleton by reacting with a functional group (a carboxyl group or a hydroxyl group, particularly, a carboxyl group) contained in the modified hyaluronic acid.

The amino group contained in the organic compound A may be any of a group represented by -NH₂ and a group represented by -NH. However, in view of better reactivity with the functional group (particularly, a carboxyl group) contained in the modified hyaluronic acid, the amino group contained in the organic compound A is preferably a group represented by -NH₂. Furthermore, it is preferable that the organic compound A has an amino group bonded to an alkylene group (—CₙH₂ₙ—, n represents an integer of equal to or greater than 1), because then the motility of the amino group can be further improved.

The molecular weight of the organic compound A is preferably equal to or greater than 140 and more preferably equal to or greater than 150, because such an organic compound is suitable for the invention of the present application owing to its reactivity that is low with respect to an unmodified hyaluronic acid and is increased in a case where a modified hyaluronic acid is used. In contrast, the molecular weight of the organic compound A is preferably equal to or less than 500 and more preferably equal to or less than 350, because then the reactivity with the modified hyaluronic acid can be ensured.

The organic compound A can further have a carboxyl group in addition to an amino group. That is, the organic compound A can be an amino acid.

In the present invention, “amino acid” refers to an organic compound having an amino group and a carboxyl group in the same molecule. In the organic compound A, a carboxyl group and an amino group may be bonded to the same carbon atom or different carbon atoms. Furthermore, the organic compound A may have both a carboxyl group bonded to a carbon atom to which an amino group is also bonded and a carboxyl group bonded to a carbon atom different from a carbon atom to which an amino group is bonded.

In a case where a carboxyl group and an amino group in the organic compound A are bonded to different carbon atoms, a carbon chain or ring can exist between the carbon atom to which the carboxyl group is bonded and the carbon atom to which the amino group is bonded. The number of carbon atoms contained in the carbon chain or ring is generally equal to or greater than 1, and preferably equal to or greater than 2. The number of carbon atoms may be equal to or less than 10 and preferably equal to or less than 6.

The organic compound A may be a compound containing an amino group and a carboxyl group and having a property in which the amino group and the carboxyl group cause self-condensation. Due to the self-condensation, an amide bond is formed. The organic compound A may have another amino group and/or carboxyl group in addition to the amino group and the carboxyl group causing self-condensation.

In the present invention, “property in which the amino group and the carboxyl group cause self-condensation” refers to a property in which, in a case where an amide bond is formed using a reagent for the amino acid synthesis described above, an amide bond is formed between the amino group and the carboxyl group present in the same molecule, or a bond is formed between different molecules of the organic compound A (an amide bond is formed between an amino group contained in one organic compound A and a carboxyl group contained in another organic compound A). According to the manufacturing method of the present embodiment, even though the organic compound A has the property of self-condensation, because the modified hyaluronic acid has a carboxyl group-terminated carboxymethyl group, the reactivity with the organic compound A is excellent. Therefore, it is possible to efficiently obtain the hyaluronic acid derivative while inhibiting the self-condensation of the organic compound A.

Examples of the organic compound A include an amino acid such as aspartic acid, lysine, glutamic acid, serine, tyrosine, valine, tryptophan, phenylalanine, tranexamic acid, arginine, y-aminobutyric acid (GABA), and levodopa, saccharides such as glucosamine and sialic acid, mexiletine, nucleic acid, and the like. Particularly, in a case where the organic compound A is, for example, tranexamic acid having a property of causing self-condensation, the self-condensation of the organic compound A can be inhibited according to the manufacturing method of the present embodiment, and hence a hyaluronic acid derivative can be efficiently obtained.

In a case where the organic compound A is an organic compound having a property of causing self-condensation, it is difficult for the compound to be bonded to a hyaluronic acid in some cases. Presumably, this is because the organic compound A causes self-condensation before reacting with the hyaluronic acid.

In contrast, according to the manufacturing method of the present embodiment, as described above, due to the carboxymethyl group of the modified hyaluronic acid, the organic compound A exhibits excellent reactivity with respect to the modified hyaluronic acid. Therefore, even though the organic compound A has a property of causing self-condensation, the reaction between the modified hyaluronic acid and the organic compound A occurs before the self-condensation. As a result, the consumption of the organic compound A due to the self-condensation can be suppressed, and hence a hyaluronic acid derivative can be efficiently obtained.

(Step of Separating)

In the manufacturing method according to the present embodiment, by the aforementioned step of reaction, a self-condensation product of the amino group and the carboxyl group in the organic compound is obtained. The manufacturing method can further include a step of separating the self-condensation product from the hyaluronic acid derivative.

In this case, examples of the step of separating include the removal of the self-condensation product by filtration and the removal by a separation treatment such as column chromatography.

(Operation and Effect)

The manufacturing method according to the present embodiment includes a step of reacting a carboxymethyl group-containing modified hyaluronic acid and/or a salt thereof with an organic compound A containing an amino group and having a molecular weight of equal to or greater than 90. Therefore, the organic compound A can be efficiently bonded to the modified hyaluronic acid and/or a salt thereof. The carboxymethyl group has a carboxyl group bonded to a methylene group (—CH₂—) on the terminal thereof, and hence, although the reason is unclear, the carboxyl group contained in the carboxymethyl group is disposed in a position further separated from the hyaluronic acid skeleton. Consequently, it is easier for the organic compound A and the modified hyaluronic acid to be closer to each other. Presumably, as a result, the organic compound A and the modified hyaluronic acid may easily react with each other, and hence the organic compound A could be efficiently bonded to the modified hyaluronic acid and/or a salt thereof.

Having a carboxymethyl group, the modified hyaluronic acid exhibits excellent reactivity with respect to an amino group. Therefore, according to the manufacturing method of the present embodiment, even though the organic compound A does not easily react with a hyaluronic acid, due to the carboxymethyl group, the reactivity with the modified hyaluronic acid is excellent. Accordingly, it is possible to efficiently obtain a hyaluronic acid derivative in which a modification rate of the organic compound A with respect to the disaccharide unit of the hyaluronic acid skeleton constituting the modified hyaluronic acid is equal to or higher than 10%.

(Hyaluronic Acid Derivative)

The hyaluronic acid derivative according to an embodiment of the present invention can be obtained by the aforementioned step of reaction. More specifically, the hyaluronic acid derivative according to the present embodiment can have a constituent unit (2) shown below. In the hyaluronic acid derivative according to the present embodiment, at least one of the groups represented by R¹, R², R³, R⁴, and R⁵ in Formula (1) that are contained in the entirety of the hyaluronic acid derivative may be substituted with a metal (for example, sodium or potassium) that belongs to Group I elements, a metal (for example, calcium, magnesium, or barium) that belongs to Group 2 elements, a metal (for example, copper, silver, or gold) that belongs to Group 11 elements, a metal (for example, zinc) that belongs to Group 12 elements, or a metal (for example, aluminum) that belongs to Group 13 elements.

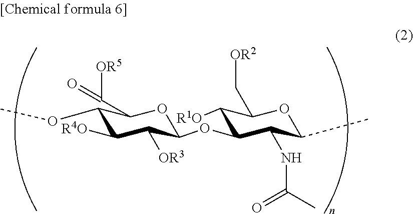

(In the formula, R¹, R², R³, R⁴, and R⁵ independently represent a hydrogen atom, a group represented by —CH₂—CO₂H, a group represented by —CH₂—CO₂⁻, or a group represented by Formula (3), and n represents a number equal to or greater than 1 and equal to or less than 7,500 (here, at least one of the groups represented by R¹, R², R³, R⁴, and R⁵ contained in the entirety of the hyaluronic acid derivative is a group represented by Formula (3).)

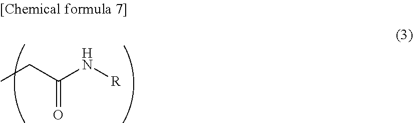

(In the formula, R represents an organic group having a molecular weight of equal to or greater than 74.)

That is, in the hyaluronic acid derivative according to the present embodiment, at least one of the constituent units constituting the hyaluronic acid derivative can be a constituent unit represented by Formula (2). At least some of R²'s in the constituent unit represented by Formula (2) are preferably a group represented by Formula (3), because then the motility of the group represented by Formula (3) is further improved.

In the hyaluronic acid derivative according to the present embodiment, the constituent unit (2) preferably contains a group represented by —CH₂—CO₂H and/or a group represented by —CH₂^{—CO}₂⁻, because then the hydrophilicity can be further improved.

More specifically, in the constituent unit represented by Formula (2) contained in the hyaluronic acid derivative according to the present embodiment, some of R²'s are preferably a group represented by Formula (3), and R² other than the group represented by Formula (3) is preferably a hydrogen atom, a group represented by —CH₂—CO₂H, or a group represented by —CH₂—CO₂⁻.

In the constituent unit represented by Formula (2), R¹, R³, R⁴, and R⁵ may each represent a hydrogen atom, a group represented by —CH₂—CO₂H, a group represented by —CH₂—CO₂⁻, or a group represented by Formula (3).

The organic group, which is represented by R in Formula (3) and has a molecular weight of equal to or greater than 74, may contain a carbon atom and a hydrogen atom and may further contain at least one kind of atom selected from an oxygen atom, a nitrogen atom, and a sulfur atom. The organic group may be chain-like or cyclic, or may have both the chain-like portion and the cyclic portion. The molecular weight of the organic group is generally equal to or less than 484, preferably equal to or greater than 124, and more preferably equal to or greater than 134.

The organic group represented by R in Formula (3) may form an amide bond through an alkylene group that R has. Examples of the alkylene group include an alkylene group having 1 to 20 carbon atoms (for example, an alkylene group having 1 to 6 carbon atoms) such as a methylene group (—CH₂—) and an ethylene group (—CH₂—CH₂—). The hyaluronic acid derivative according to the present embodiment has a hyaluronic acid skeleton and an amide bond, and an organic group is bonded to an amino group (—NH—) of the amide bond through an alkylene group (—CₙH₂ₙ—). Accordingly, the motility of the organic group can be improved.

For example, the group represented by Formula (3) may be a group derived from the organic compound A shown in Table 1 which will be described later. For example, in a case where the organic compound A in the hyaluronic acid derivative is tranexamic acid (Examples 1 to 4), by reacting an amino group contained in the tranexamic acid with a carboxyl group of the modified hyaluronic acid, a moiety derived from the tranexamic acid is bonded to the hyaluronic acid skeleton of the modified hyaluronic acid through an amide bond. In this case, the group represented by Formula (3) can be a group represented by Formula (5).

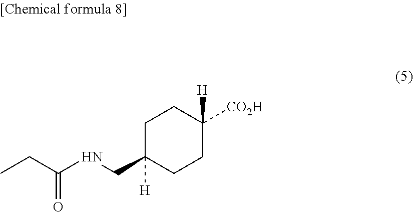

Tranexamic acid is known to have physiological activities such as whitening action, hemostatic action, anti-inflammatory action, and antiallergenic action.

The organic compound A of Examples 5 to 7 is also bonded to the hyaluronic acid skeleton of the modified hyaluronic acid by the same bonding pattern as described above.

In the hyaluronic acid derivative according to the present embodiment, a proportion (in the present specification, referred to as “modification rate” as well) of the group represented by Formula (3) contained in (bonded to) the disaccharide unit (the hyaluronic acid skeleton constituted of two sugar molecules that is represented by Formula (2)) of the hyaluronic acid skeleton can be equal to or higher than 10%, and is preferably equal to or higher than 20% and more preferably equal to or higher than 30%. In contrast, the proportion is preferably equal to or lower than 150%, and more preferably equal to or lower than 100%.

The b value, showing the hue of color, of the hyaluronic acid derivative according to the present embodiment can be equal to or greater than 0 and equal to or less than 10, and is preferably equal to or greater than 1 and equal to or less than 5. In the present invention, the b value showing the hue of color can be measured by, for example, mounting a 10 φ lens on a color-difference meter (trade name: “COLOR AND COLOR DIFFERENCE METER MODEL 1001 DP”, manufactured by NIPPON DENSHOKU INDUSTRIES Co., LTD) and covering a glass cell with 1 g of a measurement sample.

(Molecular Weight)

The molecular weight of the hyaluronic acid derivative according to the present embodiment is generally equal to or greater than 800 and equal to or less than 4,000,000. For example, the molecular weight is preferably equal to or greater than 2,000, and more preferably equal to or greater than 3,000. In contrast, the molecular weight is preferably equal to or less than 1,000,000, more preferably equal to or less than 600,000, and even more preferably equal to or less than 50,000.

(Operation and Effect)

The hyaluronic acid derivative according to the present embodiment has the constituent (2) described above. As a result, a moiety derived from the organic compound A and the disaccharide unit of the hyaluronic acid constituting the modified hyaluronic acid are bonded to each other through an amide bond. Therefore, through the hydrolysis of the amide bond in a biological body, the organic compound A or a component derived from the organic compound A can be generated. Consequently, in a case where the hyaluronic acid derivative is taken into a biological body, because the amide bond is slowly hydrolyzed, the organic compound A or a component derived from the organic compound A can be slowly released into the biological body. Therefore, the physiological activity of the organic compound A or the component derived from the organic compound A can be slowly exhibited in the biological body.

(Cosmetic Preparation)

The cosmetic preparation according to an embodiment of the present invention contain the hyaluronic acid derivative according to the present embodiment. The content of the hyaluronic acid derivative in the cosmetic preparation according to the present embodiment is equal to or greater than 0.001% by mass and equal to or less than 5% by mass for example, and can be appropriately determined according to the form of use.

The aspect of the cosmetic preparation according to the present embodiment is not particularly limited, and examples thereof include skin care cosmetic preparation. In a case where the hyaluronic acid derivative according to the aforementioned embodiment is used in the skin care cosmetic preparation, the organic compound A (or a component derived from the organic compound A) contained in the hyaluronic acid derivative is slowly separated from the hyaluronic acid derivative in a biological body. Therefore, the cosmetic preparation have an excellent property in which the action of the organic compound A (or a component derived from the organic compound A) is slowly exhibited, and have appropriate viscosity and a strong water retention effect. Accordingly, the cosmetic preparation can moisturize the skin and improve the feeling of roughness of the skin.

Examples of the aspect of the skin care cosmetic preparation according to the present embodiment include a facial cleanser, a washer, a toner (for example, a whitening toner), a cream (for example, a vanishing cream and a cold cream), an emulsion, an essence, a mask pack (for example, a gel-like peel-off type, a paste-like wipe-off type, and a powder-like wash-off type), a cleanser, a foundation, a lipstick, a lip cream, a lip-gloss, a lip liner, a blusher, a shaving lotion, an after sun lotion, a deodorant lotion, a body lotion (including a hand care lotion and a foot care lotion), a body oil, a soap, and a bathing agent.

The following components may be additionally formulated with the cosmetic preparation according to the present embodiment. Examples of the components include cationized polysaccharides (for example, cationized hyaluronic acid, cationized hydroxyethyl cellulose, cationized guar gum, cationized starch, cationized locust bean gum, cationized dextran, cationized chitosan, and cationized honey), an anionic surfactant (for example, alkylbenzene sulfonate, a polyoxyalkylene alkyl ether sulfuric acid ester salt, an alkyl sulfuric acid ester salt, olefin sulfonate, a fatty acid salt, and dialkyl sulfosuccinate), a nonionic surfactant (for example, a polyoxyethylene fatty acid ester and a polyoxyethylene hydrogenated castor oil derivative), a cationic surfactant (for example, an alkyl trimethyl ammonium salt, a dialkyl dimethyl ammonium salt, an alkyl pyridinium salt, and stearyl trimethyl ammonium chloride), an amphoteric surfactant (for example, alkyl betaine, alkyl amidopropyl betaine, imidazolinium betaine, egg yolk lecithin, and soybean lecithin), oil (for example, silicone, a silicone derivative, liquid paraffin, squalane, beeswax, carnauba wax, olive oil, avocado oil, camellia oil, jojoba oil, and horse oil), a moisturizer (for example, sodium hyaluronate, hydrolyzed hyaluronic acid, acetylated hyaluronic acid, dimethylsilanol hyaluronate, ceramide, diphytosteryl octyldodecyl lauroyl glutamate, phytoglycogen, a hydrolyzed eggshell membrane, trehalose, glycerin, atelocollagen, sorbitol, maltitol, and 1,3-butylene glycol), a higher fatty acid (for example, lauric acid, behenic acid, palmitic acid, stearic acid, isostearic acid, and oleic acid), a higher alcohol (for example, cetyl alcohol, stearyl alcohol, behenyl alcohol, isostearyl alcohol, and batyl alcohol), a polyol (for example, glycerin, diglycerin, 1,3-propanediol, propylene glycol, polyethylene glycol, and pentylene glycol), a thickener (for example, cellulose ether, a carboxyvinyl polymer, xanthan gum, and dextrin palmitate), an amphoteric polymer resin compound (for example, betainated dialkylaminoalkyl acrylate copolymer), a cationic polymer resin compound (for example, a cationized vinyl pyrrolidone/dimethylaminoethyl methacrylate copolymer and a polydimethyldiallyl ammonium halide-type cationic polymer), antiseptics (for example, methyl paraben, ethyl paraben, butyl paraben, propyl paraben, and phenoxyethanol), an antioxidant (for example, tocopherol and BHT), a sequestrant (for example, edetate and etidronate), an ultraviolet absorber (for example, a benzophenone derivative, a p-aminobenzoic acid derivative, and a methoxycinnamic acid derivative), an ultraviolet reflective agent (for example, titanium oxide and zinc oxide), a protein hydrolysate (for example, a gelatin peptide, a collagen peptide, a soybean peptide, a wheat peptide, a milk peptide, a silk peptide, and an egg white peptide), an amino acid (for example, arginine, glutamic acid, glycine, alanine, hydroxyproline, cysteine, serine, and L-theanine), a natural substance extract (a sophora root extract, a chamomile extract, a seaweed extract, a eucalyptus extract, a royal jelly extract, a rosemary extract, and a beech tree extract), other functional components (coenzyme Q10, arbutin, polyquaternium 51, elastin, platinum nanocolloide, retinol palmitate, panthenol, allantoin, sodium dilauroyl glutamate lysine, magnesium ascorbyl phosphate, L-ascorbic acid 2-glucoside, ellagic acid, kojic acid, linoleic acid, and tranexamic acid), a phospholipid polymer, aromatics, and colorants.

[Food Composition]

The food composition according to an embodiment of the present invention contains the hyaluronic acid derivative according to the aforementioned embodiment. The content of the hyaluronic acid derivative in the food composition according to the present embodiment is equal to or greater than 0.001% by mass and equal to or less than 5% by mass for example, and can be appropriately determined according to the form of use. In a case where the hyaluronic acid derivative according to the aforementioned embodiment is used in the food composition, the organic compound A (or a component derived from the organic compound A) contained in the hyaluronic acid derivative is slowly separated from the hyaluronic acid derivative in a biological body. Therefore, the food composition has an excellent property in which the action of the organic compound A (or a component derived from the organic compound A) is slowly exhibited. In addition, the food composition has excellent texture because the viscosity thereof is lower than hyaluronic acid having a molecular weight that is approximately equivalent to that of the composition.

The aspect of the food composition containing the hyaluronic acid derivative according to the present embodiment is not particularly limited, and examples thereof include all of general food such as processed food from rice as staple food, bakery products, retort canned food as side dish, frozen food, side dishes, dehydrated food, seasonings such as mayonnaise, beverages, cake and confectionary, desserts, and liquid, gel-like, and soft capsule-like supplements and all of the specific healthcare food permitted to perform physiological functions.

[Pharmaceutical Composition]

The pharmaceutical composition according to an embodiment of the present invention contains the hyaluronic acid derivative according to the aforementioned embodiment. The content of the hyaluronic acid derivative in the pharmaceutical composition according to the present embodiment is equal to or greater than 0.001% by mass and equal to or less than 5% by mass for example, and can be appropriately determined according to the form of use. In a case where the hyaluronic acid derivative according to the aforementioned embodiment is used in the pharmaceutical composition, the organic compound A (or a component derived from the organic compound A) contained in the hyaluronic acid derivative is slowly separated from the hyaluronic acid derivative in a biological body. Therefore, the pharmaceutical composition has an excellent property in which the action of the organic compound A (or a component derived from the organic compound A) is slowly exhibited.

The form of use of the pharmaceutical composition according to the present embodiment is not particularly limited, and the pharmaceutical composition can be used in the form of powder, granules, a high-concentration liquid, a low-concentration liquid, and the like. Considering the stability of the molecular weight of the hyaluronic acid derivative according to the aforementioned embodiment, the pharmaceutical composition in a dehydrated form is more preferred than a liquid form.

If necessary, an extender, a binder, a lubricant, a preservative, an antioxidant, aromatics, a sweetener, an acidulant, an excipient, and the like can be formulated with the pharmaceutical composition according to the present embodiment. Furthermore, various nutritional components including vitamins such as vitamin C, vitamin B2, vitamin B12, and vitamin E, nutritional components such as nucleic acid, chondroitin sulfate, and collagen, and mineral components such as iron and zinc can also be formulated with the pharmaceutical composition.

### EXAMPLE

Hereinafter, the present invention will be more specifically described based on examples, but the present invention is not limited to the following examples.

### Preparation Example:

### Method for Manufacturing Modified Hyaluronic Acid Containing Carboxymethyl Group

2.3 g of sodium hydroxide was weighed into a 30 mL sample vial, and then 6 mL of water was added thereto for dissolution. Thereafter, 2.0 g of raw material hyaluronic acid (molecular weight: about 10,000) was added thereto and dissolved, 3.6 g of monobromoacetic acid was then added thereto and dissolved, and then the solution was left to stand for 40 hours at room temperature. The pH of the reaction solution was 13. Then, 80 mL of ethanol was put into a 200 mL beaker, and the aforementioned reaction solution was added thereto with stirring such that a carboxymethyl group-containing modified hyaluronic acid was precipitated. Thereafter, the precipitate was recovered into a 200 mL beaker by using 400 mesh filter cloth, and then 40 mL of a 10% aqueous sodium chloride solution was added thereto such that the precipitate dissolved. The pH of the solution was adjusted using a 8% aqueous hydrochloric acid solution, and 80 mL of ethanol was then added to the solution with stirring, thereby causing reprecipitation of a modified hyaluronic acid containing a carboxymethyl group. The solution was washed three times with 100 mL of 80% water-containing ethanol, filtered under reduced pressure, and dried under reduced pressure for 3 hours at 55° C., thereby obtaining a carboxymethyl group-containing modified hyaluronic acid of Example 1.

Carboxymethyl group-containing modified hyaluronic acids of Examples 2 to 7 and Comparative Example 1 were obtained by the same method as in Example 1, except that the molecular weight of the raw material hyaluronic acid, the amount of water used, the amount of sodium hydroxide used, the reaction temperature, and the reaction time were changed. More specifically, the molecular weight of the raw material hyaluronic acid used in each of Examples 2 to 7 and Comparative Example 1 is as shown in Table 1. In Examples 3, 4, 5, and 7 and Comparative Example 1, the amount of water used was 12 mL, and the amount of sodium hydroxide used was 2.1 g. Furthermore, in Examples 3, 4, 5, and 7 and Comparative Example 1, the reaction temperature was 4° C. In addition, in Examples 3, 4, 5, and 7 and Comparative Example 1, the reaction time was 24 hours, 24 hours, 6 hours, 24 hours, and 6 hours respectively. In the present example, “room temperature” means a temperature equal to or higher than 20° C. and equal to or lower than 30° C.

### Example 1:

### Method for Manufacturing Hyaluronic Acid Derivative

100 mL of pure water was put into a 500 mL beaker, and then the raw material hyaluronic acid having the molecular weight shown in Table 1 or the raw material modified hyaluronic acid (carboxymethyl group-containing modified hyaluronic acid) having the molecular weight shown in Table 1 was added thereto in an amount of 1 g (2.5 mmol) and then dissolved. Subsequently, 480 mg (2.5 mmol) of EDC·HCl was added thereto, 432 mg (2.75 mmol) of tranexamic acid was then added thereto as the organic compound A, and the solution was reacted for 3 hours at room temperature (25° C.). Furthermore, 432 mg (2.75 mmol) of tranexamic acid was further added thereto, and the solution was reacted overnight at room temperature. Thereafter, 5 g of sodium chloride was added thereto, 200 mL of ethanol was then added thereto, and then the solution was left to stand. Then, the supernatant was removed by decantation. Furthermore, the solution was washed twice with 100 mL of an 80% aqueous ethanol solution and with 100 mL of ethanol, and then the obtained residue was dried, thereby obtaining a hyaluronic acid derivative according to Example 1.

### Examples 2 to 7 and Comparative Example 1

Hyaluronic acid derivatives according to Examples 2 to 7 and Comparative Example 1 of the present application were obtained by the same method as in Example 1, except that the type of the raw hyaluronic acid, the raw material modified hyaluronic acid, and the organic compound A used was changed as shown in Table 1 of the present application.

TABLE 1

| | | | | Raw material | Raw material modified | |
|---|---|---|---|---|---|---|
| | | | | hyaluronic acid | hyaluronic acid | |
| | | | | Molecular weight of | Molecular | Carboxy- |
| | Organic compound A | | | raw hyaluronic acid | weight or raw | methylation rate of |
| | | | Molec- | as raw material of | material modified | raw material |
| | | | ular | raw material modified | hyaluronic | modified hyaluronic |
| | Name | Structural formula | weight | hyaluronic acid | acid | acid |
| Example 1 | Tranexamic acid | 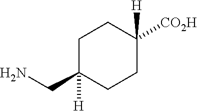 [SMILES: NC[C@H]1CC[C@H](C(=O)O)CC1] | 157 | 10,000 | About 10,000 | 77% |
| Example 2 | Tranexamic acid | 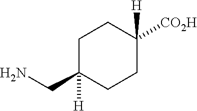 [SMILES: NC[C@H]1CC[C@H](C(=O)O)CC1] | 157 | 10,000 | About 10,000 | 46% |
| Example 3 | Tranexamic acid | 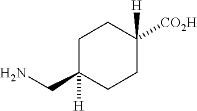 [SMILES: NC[C@H]1CC[C@H](C(=O)O)CC1] | 157 | 300,000 | About 150,000 | 88% |
| Example 4 | Tranexamic acid | 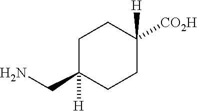 [SMILES: NC[C@H]1CC[C@H](C(=O)O)CC1] | 157 | 1,700,000 | 1,050,000 | 98% |
| Example 5 | GABA | 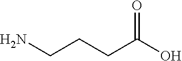 | 103 | 1,700,000 | 1,290,000 | 68% |
| Example 6 | Arginine | 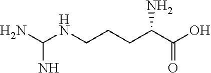 [SMILES: NC(N)NCCC[C@H](N)C(=O)O] | 174 | 10,000 | About 10,000 | 68% |
| Example 7 | Levodopa | 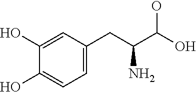 [SMILES: N[C@@H](Cc1ccc(O)c(O)c1)C(O)O] | 197 | 1,700,000 | 1,050,000 | 98% |
| Compara- tive Example 1 | Glycine | 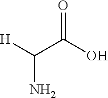 | 75 | 1,700,000 | 1,290,000 | 68% |

| | Hyaluronic acid derivative | | | | | |
|---|---|---|---|---|---|---|
| | Modification rate | | | | | |
| | 1) Modification rate | Molecular weight | 2) Modification | | | |
| | in derivative with | of raw material | rate in derivative | | Transmittance | |
| | raw material | hyaluronic acid | with raw | Rate of | Transmittance | Transmittance |
| | hyaluronic | as raw material | material modified | increase = | of reaction | of reaction |
| | acid (%) | of derivative in 1) | hyaluronic acid (%) | 100 × 2)/1) | solution in 1) | solution in 1) |
| Example | 3.8 | About 10,000 | 42.7 | 1124% | 59% | 99% |
| 1 | | | | | | |
| Example | 3.8 | About 10,000 | 26.4 | 695% | 59% | 74% |
| 2 | | | | | | |
| Example | 11.5 | 140,000 | 37.8 | 329% | 15% | 99% |
| 3 | | | | | | |
| Example | 12.3 | 1,620,000 | 61.7 | 502% | 80% | 95% |
| 4 | | | | | | |
| Example | 32 | 1,230,000 | 86 | 271% | 94% | 95% |
| 5 | | | | | | |
| Example | 24 | About 10,000 | 41 | 171% | 99% | 99% |
| 6 | | | | | | |
| Example | 5.4 | 1,200,000 | 9.1 | 169% | 15% | 12% |
| 7 | | | | | | |
| Compara- | 31 | 1,230,000 | 39 | 127% | 96% | 97% |
| tive | | | | | | |
| Example | | | | | | |
| 1 | | | | | | |

The modification rate and the transmittance shown in Table 1 are values measured by the following method.

(Method for Measuring Modification Rate)

In the hyaluronic acid derivatives according to examples and comparative examples described above, a proportion (modification rate: %) of the organic compound A contained in the disaccharide unit of the hyaluronic acid skeleton is a value obtained by performing ¹H-NMR spectroscopy on a 1% by mass aqueous solution of the hyaluronic acid derivatives of the present examples by the following method.

<Measurement of Modification Rate by ¹H-NMR>

Sample concentration: 1.0% (D₂O)

Device: Varian NM system 400NB model (Varian Technology Japan Limited)

Observation frequency: 400 MHz

Temperature: 30° C.

Standard: DSS (0 ppm)

Pulse width: 45°

Integration frequency: 64

In the case of Examples 1, 2, 3, and 4, in the ¹H-NMR spectrum, a peak that appears at around 3.4 ppm is considered to be a peak showing one proton in the hyaluronic acid skeleton, and a peak that appears at around 1.0 ppm is considered to be a peak showing two protons (-NH₂) contained in the structure derived from the tranexamic acid which is the organic compound A. From these two peaks, based on the following equation, the proportion (modification rate (%)) of tranexamic acid bonded to the disaccharide unit of the hyaluronic acid skeleton was calculated.

For each of the hyaluronic acid derivatives of Examples 5 to 7 and Comparative Example 1, from the integration value of the peaks derived from the hyaluronic acid and each of the organic compounds and the number of protons, the modification rate was calculated. More specifically, in the hyaluronic acid derivative of Example 5, a peak showing two protons (-NH₂) contained in the structure derived from GABA as the organic compound A appeared at around 1.8 ppm. In the hyaluronic acid derivative of Example 6, a peak showing two protons (-NH₂) contained in the structure derived from arginine as the organic compound A appeared at around 1.7 ppm. In the hyaluronic acid derivative of Example 7, a peak showing one proton (—NH—) contained in the structure derived from levodopa as the organic compound A appeared at around 6.9 ppm. In the hyaluronic acid derivative of Comparative Example 1, a peak showing two protons (—NH₂—) contained in the structure derived from glycine appeared at around 3.6 ppm.

Modification rate (%)=(integration value of predetermined peak derived from organic compound A/number of protons of predetermined peak derived from organic compound A)/(integration value of predetermined peak derived from hyaluronic acid/number protons of predetermined peak derived from hyaluronic acid)×100

(Transmittance)

The transmittance of the reaction solution shown in Table 1 is a parameter showing to what extent the self-condensation product of the organic compound A is generated. More specifically, the absorbance of a 1% by mass aqueous solution of each of the hyaluronic acid derivatives obtained in the aforementioned examples and comparative example for light at a wavelength of 660 nm is measured using a spectrophotometer UV-2440 (model name, manufactured by Shimadzu Corporation), and the transmittance is expressed as a value determined in a case where the absorbance of the aqueous solution to which the hyaluronic acid derivative is not yet added is regarded as being 100%. Presumably, the lower the transmittance of the reaction solution, the higher the turbidity of the aqueous solution, and the larger the amount of the self-condensation product generated.

As is evident from Table 1, because the manufacturing method according to the present embodiment includes a step of reacting a carboxymethyl group-containing modified hyaluronic acid and/or a salt thereof with an organic compound containing an amino group and having a molecular weight of equal to or greater than 90, as shown in the Examples 1 to 7 of the present invention, compared to the case where the raw material hyaluronic acid is used, a hyaluronic acid derivative is obtained in which the modification rate is more efficiently increased (according to Examples 1 to 7 shown in Table 1, in a case where the raw material modified hyaluronic acid is used, the modification rate increases further by a rate of about equal to or greater than 130% (more specifically, 169% to 1,124%) than in a case where the raw material hyaluronic acid is used).

In contrast, in Comparative Example 1 of the present application, because the organic compound (glycine) having a molecular weight of less than 90 was used, the modification rate was only slightly higher (increased by 127%) in a case where the raw material modified hyaluronic acid was used than in a case where the raw material hyaluronic acid was used.

1. A method for manufacturing a hyaluronic acid derivative, comprising:
a step of reacting a carboxymethyl group-containing modified hyaluronic acid and/or a salt thereof with an organic compound containing an amino group and having a molecular weight of equal to or greater than 90.

2. The method for manufacturing a hyaluronic acid derivative according to claim 1,
wherein the organic compound further contains a carboxyl group.

3. The method for manufacturing a hyaluronic acid derivative according to claim 2,
wherein in the organic compound, the amino group and the carboxyl group are bonded to different carbon atoms.

4. The method for manufacturing a hyaluronic acid derivative according to claim 1,
wherein in the organic compound, the amino group is bonded to an alkylene group.

5. The method for manufacturing a hyaluronic acid derivative according to claim 1,
wherein the carboxymethyl group-containing modified hyaluronic acid and/or a salt thereof have a constituent unit (1) shown below, 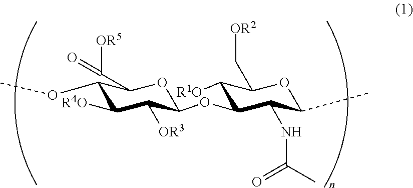
wherein R¹, R², R³, R⁴, and R⁵ independently represent a hydrogen atom, a group represented by —CH₂—CO₂H, or a group represented by —CH₂—CO₂, and n represents a number equal to or greater than 1 and equal to or less than 7,500 and
a case is excluded where all of R¹, R², R³, R⁴, and R⁵ in the entirety of the carboxymethyl group-containing modified hyaluronic acid and/or a salt thereof represent a hydrogen atom.

6. The method for manufacturing a hyaluronic acid derivative according to claim 1,
wherein a carboxymethylation rate with respect to a disaccharide unit constituting the carboxymethyl group-containing modified hyaluronic acid and/or a salt thereof is equal to or higher than 5% and equal to or lower than 200%.

7. The method for manufacturing a hyaluronic acid derivative according to claim 1,
wherein the amino group contained in the organic compound is a group represented by -NH₂.

8. A hyaluronic acid derivative, comprising:
a constituent unit (2) shown below, 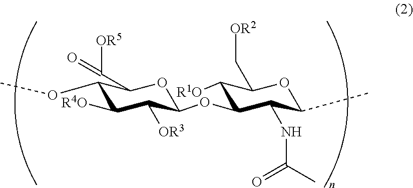
wherein R¹, R², R³, R⁴, and R⁵ independently represent a hydrogen atom, a group represented by —CH₂—CO₂H, a group represented by —CH₂—CO₂, or a group represented by Formula (3), and n represents a number equal to or greater than 1 and equal to or less than 7,500, and
at least one of the groups represented by R¹, R², R³, R⁴, and R⁵ contained in the entirety of the hyaluronic acid derivative is a group represented by Formula (3), 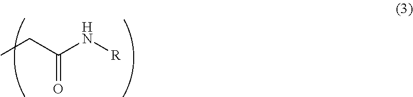
wherein R represents an organic group having a molecular weight of equal to or greater than 74.

9. The hyaluronic acid derivative according to claim 8,
wherein a proportion of the group represented by Formula (3) contained in a disaccharide unit of a hyaluronic acid skeleton constituting the constituent unit (2) is equal to or higher than 10%.

10. The hyaluronic acid derivative according to claim 8,
wherein the constituent unit (2) contains a group represented by —CH₂—CO₂H and/or a group represented by —CH₂—CO₂.

11. (canceled)

12. A cosmetic preparation, comprising the hyaluronic acid derivative according to claim 8.

13. A food composition, comprising the hyaluronic acid derivative according to claim 8.

14. A pharmaceutical composition, comprising the hyaluronic acid derivative according to claim 8.
